(19) United States
(12) Patent Application Publication
(10) Pub. No.: US 2026/0031315 A1
(43) Pub. Date: Jan. 29, 2026

(54) SYSTEMS AND METHODS OF ELECTROPHORESIS-CORRELATIVE (ECO) MASS SPECTROMETRY (MS)

(71) Applicant: University of Maryland, College Park, College Park, MD (US)

(72) Inventors: Peter Nemes, Silver Spring, MD (US); Bowen Shen, Greenbelt, MD (US)

(21) Appl. No.: 19/343,881

(22) Filed: Sep. 29, 2025

Related U.S. Application Data
(63) Continuation of application No. PCT/US2024/023335, filed on Apr. 5, 2024.
(60) Provisional application No. 63/494,747, filed on Apr. 6, 2023.

(51) Int. Cl. H01J 49/16; G01N 33/68; H01J 49/00; H01J 49/40; H01J 49/42

(52) U.S. Cl. CPC H01J 49/167; G01N 33/6848; H01J 49/0031; H01J 49/40; H01J 49/425

(57) ABSTRACT

Systems and methods for specialized data acquisition in capillary electrophoresis electrospray ionization mass spectrometry (MS) that includes (a) mass-to-charge (m/z) vs. migration time (MT) correlation, (b) ion mobility (IM) vs. MT correlation, and (c) m/z vs. IM vs. MT correlation to advance molecular analysis via data-dependent, data-independent, and targeted analysis methods executed on mass spectrometers using diverse types of mass analyzers, including but not limited to orbitrap, time-of-flight, and ion mobility time-of-flight mass analyzers. Electrophoresis-correlative (Eco) MS enhances the detection, identification, and quantification of molecules, as is demonstrated here for complex proteome samples.

### CROSS REFERENCE TO RELATED APPLICATIONS

This is a continuation patent application which claims priority under 35 U.S.C. § 120 to Patent Cooperation Treaty (PCT) Application PCT/US2024/023335, filed Apr. 5, 2024, which is hereby incorporated by reference in its entirety, including without limitation, the specification, claims, and abstract, as well as any figures, tables, appendices, or drawings thereof.

This application claims priority under 35 U.S.C. § 119 to provisional patent application U.S. Ser. No. 63/494,747, filed Apr. 6, 2023. The provisional patent application is herein incorporated by reference in its entirety, including without limitation: the specification, claims, and abstract, as well as any figures, tables, appendices, or drawings thereof.

### TECHNICAL FIELD

The subject matter of the present disclosure generally relates to the identification and quantification of molecules using mass spectrometry.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Mass spectrometry (MS) is the leading technology to identify and quantify biomolecules based on the detection of their mass-to-charge (m/z) values, akin to weighing molecules. MS is the primary driver of proteomics, peptidomics, and metabolomics, as well as post-translational modifications. MS can accomplish the quantification of proteomes using only limited amounts of starting materials, including even single cells. MS can also be used for transcriptomics and genomics detection.

Although hundreds to thousands of proteins were successfully identified from microgram to nanogram level of protein digest, the key limitation for protein identifications arises from the limited number of tandem MS that can be performed due to the speed of current mass spectrometers. Thus, there exists a need in the art for an apparatus that can increase the speed of mass spectrometers.

Recent inventions have also made MS capable of separating molecules further in the gas phase via a process called ion mobility MS so that molecules can be better analyzed. How the mass spectrometer is operated fundamentally determines the types and numbers of different molecules that can be detected. Scientists are in pursuit in better ways to operate mass spectrometers to detect and quantify more molecules, better, at higher sensitivity. Thus, there is also a high and still unmet need to develop new tools and ways to operate mass spectrometers to better report on molecules.

To facilitate MS detection, biomolecules are usually separated first in the solution phase so that these molecules can be slowly introduced, one after the other ideally, for the mass spectrometer to detect them. Because mass spectrometers can identify only so many molecules at a time due to limitation in their duty cycle, separation dramatically enhances the depth of molecular coverage. Currently, the mainstream technology for separation is liquid chromatography (LC), which retains molecules based on their hydrophobic/hydrophilic characteristics on a stationary phase. Capillary electrophoresis (CE) is another separation technology, which separates molecules based on a completely different principle. CE separates molecules (ions) in solution based on their electrophoretic mobility under an electric field. The electrophoretic mobility of a molecule is known to depend on its size, charge, and hydration shell, among other factors.

### SUMMARY

Systems and methods described herein are directed to electrophoresis-correlative mass spectrometry (Eco-MS). Example systems and methods show m/z-dependent separation in capillary electrophoresis (CE) allows deeper utilization of the limited duty cycle of tandem mass spectrometry. Example systems and methods also use m/z-predictive ion separation to boost the economy of MS proteomics on orbitrap and trapped ion mobility time-of-flight (timsTOF) analyzer instruments. Using HeLa digests and single embryonic stem cells, embodiments of Eco-MS in the present disclosure provide notable improvements in the detectable proteome.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to leverage the principle of capillary electrophoresis (CE) in the separation of biomolecules in order to operate a mass spectrometer in a smart, intelligent way and advance the analysis of biomolecules.

It is still yet a further object, feature, and/or advantage of the present disclosure to control the way the mass spectrometer measures the molecules so that these molecules can be better analyzed. Based on lab data, the m/z (size) of the molecules correlates with the electrophoretic mobility of the molecule, which essentially translates into time the molecule takes to separate a given length in a capillary. This correlation CE is unique in that the molecules form entire trends in their m/z vs. separation time (also known as migration time in CE). This correlation does not exist in liquid chromatography (LC), where molecules with different m/z values separate randomly. Therefore, CE allows for predicting what types of molecules separate when (at what separation times) and with what m/z value. CE also allows for prediction of m/z values if the electrophoretic mobility of the molecule is known.

It is still yet a further object, feature, and/or advantage of the present disclosure to expand the detection and quantification of broad types of molecules. This embodiment can enhance the detection of broad types of molecules, including but not limited to proteins, metabolites, peptides, transcripts, and genes.

It is still yet a further object, feature, and/or advantage of the present disclosure to enable ultrasensitive proteomics using electrophoresis-correlative mass spectrometry.

It is still yet a further object, feature, and/or advantage of the present disclosure to allow deeper utilization of the limited duty cycle of tandem mass spectrometry.

It is still yet a further object, feature, and/or advantage of the present disclosure to improve detection sensitivity, duty cycle, quantification, and the depth of coverage for the 'omes.

It is still yet a further object, feature, and/or advantage of the present disclosure to know and predict when particular m/z values need detection.

It is still yet a further object, feature, and/or advantage of the present disclosure to tailor the mass spectrometer to detect the specific m/z values or regions of m/z values that need analysis.

It is still yet a further object, feature, and/or advantage of the present disclosure to focus the mass spectrometer's bandwidth to what the mass spectrometer needs to detect.

It is still yet a further object, feature, and/or advantage of the present disclosure to aid multiple modalities of MS operation, including but not limited to bottom-up, middle-down, and top-down proteomics.

Systems and methods of electrophoresis-correlative (ECO) mass spectrometry (MS) disclosed herein can be used in a wide variety of applications. For example, example embodiments of the present disclosure are demonstrated for peptides from a protein digest via bottom-up proteomics, where peptides are sequenced. In yet another example, embodiments of the present disclosure can be implemented on any mass spectrometer, ranging from quadrupole to ion trap to time-of-flight to orbitrap mass spectrometers, which are leading forms of analyzers in modern mass spectrometer.

It is still yet a further object, feature, and/or advantage of the present disclosure to use the migration-predictive correlation to operate the mass spectrometer smarter via already existing identification strategies, including but not limited to data-dependent (DDA) and data-independent analysis (DIA).

It is still yet a further object, feature, and/or advantage of the present disclosure to use an algorithm during DDA to predict separation times, either dynamically or statically, so that the mass spectrometer can be programmed to only analyze the ions that need analysis via the identification strategies.

It is still yet a further object, feature, and/or advantage of the present disclosure to predict a range of m/z is targeted for analysis using DIA.

It is still yet a further object, feature, and/or advantage of the present disclosure to utilize the correlation of electrophoretic migration in CE in the liquid phase with gas-phase ion mobility separation (collision cross sections-CCS from measured 1/k0 values). For example, knowledge of this information would allow mass spectrometers equipped with ion mobility to predict what ion mobility values (e.g., in DDA operation) or ranges of values (e.g., in DIA operation) need analysis so as to enhance the bandwidth, sensitivity, duty cycle, and quantification of molecules. This embodiment can be realized on any mass spectrometer that has an ion mobility cell, regardless of the vendor of the instrument.

It is still yet a further object, feature, and/or advantage of the present disclosure to yield a unique scenario where both the m/z and the ion mobility characteristic (e.g., 1/k0 or CCS values) are tailored, thus vastly improving the operation of the mass spectrometer.

Embodiments described herein can be implemented on mass spectrometers that are equipped with an ion mobility cell.

It is still yet a further object, feature, and/or advantage of the present disclosure to detect dark 'omes, viz. molecules that have been difficult to detect.

It is still yet a further object, feature, and/or advantage of the present disclosure to use a mass spectrometer in combination with in silico or experimental prediction of the types of molecules that need analysis from knowledge bases, such as protein sequences and the resulting peptides in bottom-up/middle-down, so that the mass spectrometer can also detect molecules that it otherwise would be challenging to measure.

It is still yet a further object, feature, and/or advantage of the present disclosure to include a mechanism in a mass spectrometer that combats lower separation time reproducibility in LC.

It is still yet a further object, feature, and/or advantage of the present disclosure to include a mechanism in a mass spectrometer that actively monitor molecules (e.g., peptides) that separate in CE to predict the next molecules or ranges of molecules that are anticipated to separate. The mass spectrometer can thus operate on the fly, without requiring stringent separation reproducibility during separation, thereby eliminating traditional issues.

It is preferred that the improved mass spectrometers described herein be safe, cost effective, accurate, and durable. For example, the housing of the mass spectrometer can be adapted to resist excessive heat, static buildup, corrosion, and/or mechanical failures (e.g. cracking, crumbling, shearing, creeping) due to excessive impacts and/or prolonged exposure to tensile and/or compressive forces acting on the mass-spectrometer.

Methods can be practiced which facilitate use, manufacture, assembly, maintenance, and repair of a mass spectrometer which accomplishes some or all of the previously stated objectives.

Mass spectrometers can be incorporated into systems, such as ultrasensitive high resolution mass spectrometry (HRMS) platform(s), which accomplish some or all of the previously stated objectives.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a schematic diagram of Eco-MS applied on a CE-MS instrument. The generated ions are detected and quantified in the mass spectrometer.

FIGS. 2A-2B are schematic diagram of detecting ESI-generated ions using quadrupole mass spectrometers with different principles of operation: FIG. 2A is without ion mobility (IM) separation, illustrating an orbitrap analyzer (Thermo Scientific, Waltham, MA); and FIG. 2B is with IM separation, illustrating a TOF analyzer (Bruker Daltonics, Billerica, MA).

FIG. 3 is a flowchart showing Eco-MS applied for untargeted and targeted analyses using CE-MS without and with IM separation.

FIGS. 4A-4B presents examples of correlations. FIG. 4A presents m/z vs. migration time (MT) forming trends. FIG. 4B presents IM vs. MT trends for 1 ng and 200 pg of HeLa cell proteome digest, respectively. Charge states for the detected peptides are annotated.

FIG. 5 presents a flow-chart for the integration of capillary electrophoresis (CE) separation, electrospray ionization (ESI), and Eco-MS-enhanced identification/quantification of molecular ions by intelligent operation of the mass spectrometer.

FIGS. 6A-6D presents enhanced protein identification and quantification using DIA on an orbitrap mass spectrometer measuring 1 ng of HeLa proteome digest. FIG. 6A show examples for m/z vs. MT frames. FIG. 6B shows a comparison of protein identifications between classical DIA and Eco-MS DIA. FIG. 6C shows a comparison of classical DIA and Eco-MS DIA for a quantitative reproducibility. FIG. 6D shows a dynamic concentration range of the measured proteins.

FIGS. 7A-7D presents enhanced protein identification and quantification using IM-DDA on an IM-TOF mass spectrometer measuring 500 pg of HeLa proteome digest. FIG. 7A shows examples for IM vs. MT frames. FIG. 7B shows a comparison of protein identifications between classical IM-DDA and Eco-MS IM-DDA. FIG. 7C shows a comparison of classical IM-DDA and Eco-MS IM-DDA for quantitative reproducibility.

FIG. 7D shows a comparison of classical IM-DDA and Eco-MS IM-DDA for the dynamic concentration range of the measured proteins.

FIGS. 8A-8C present enhanced protein identification and quantification using IM-DIA on an IM-TOF mass spectrometer measuring 500 pg of HeLa proteome digest. FIG. 8A provides examples for IM vs. MT frames. The m/z vs. migration time frame is not shown (see example in FIG. 6A on 1 ng of proteome digest). FIG. 8B shows a comparison of protein identifications between classical IM-DIA and Eco-MS IM-DIA. FIG. 8C shows a comparison of classical IM-DIA and Eco-MS IM-DIA for quantitative reproducibility. FIG. 8D shows a comparison of classical IM-DIA and Eco-MS IM-DIA for the dynamic concentration range of the measured proteins.

FIG. 9 graphs peptide mass vs. separation time for CE-μESI (top) and nLC-nESI (bottom). Regarding predictable CE separation, FIG. 10 graphs calculated electrophoretic mobility of charge vs. electrophoretic mobility of charge.

FIG. 11A graphs data-dependent analysis (DDA): specifically: m/z vs. migration time correlation for the proteome digest HeLa and the +3 charge state.

FIG. 11B graphs data-independent analysis (DIA): specifically: m/z vs. migration time correlation for the proteome digest HeLa and the +3 charge state.

FIG. 12A graphs mobility value vs. m/z for +1, +2, +3, and +4 charge states and FIG. 12B graphs m/z vs. mobility values for +1, +2, +3, and +4 charge states (CE-μESI: 1,127 PEPTIDES (20 NG)).

FIG. 13A graphs target ion mobility vs migration time correlation for the proteome digest HeLa and the +3 charge state.

FIG. 13B graphs data-independent analysis (DIA): specifically: target ion mobility vs migration time correlation for the proteome digest HeLa and the +3 charge state.

FIG. 14A graphs mobility value vs. m/z for +1, +2, +3, and +4 charge states.

FIG. 14B graphs m/z vs. mobility values for +1, +2, +3, and +4 charge states.

FIG. 15 graphs the mobility values vs migration time for the +3 charge state.

FIGS. 16A-16C graph measured CCS vs predicted drift time for the +2 charge state (FIG. 16A), the +3 charge state (FIG. 16B), and the +4 charge state (FIG. 16C).

FIGS. 17A-17B graph mass vs migration time (FIG. 17A) and mobility values vs. mass (FIG. 17B) for +1 charge states.

FIGS. 18A-18B graph mass vs migration time (FIG. 18A) and mobility values vs. mass (FIG. 18B) for +2 charge states.

FIGS. 19A-19B graph mass vs migration time (FIG. 19A) and mobility values vs. mass (FIG. 19B) for +3 charge states.

FIGS. 20A-20D graph calculated estimated mobility vs expected mobility values for each charge state.

FIG. 20A graphs calculated electrophoretic mobility of charge +1 as a function (linear fit) of electrophoretic mobility of charge +1.

FIG. 20B graphs calculated electrophoretic mobility of charge +2 as a function (linear fit) of electrophoretic mobility of charge +2.

FIG. 20C graphs calculated electrophoretic mobility of charge +3 as a function (linear fit) of electrophoretic mobility of charge +3.

FIG. 20D graphs calculated electrophoretic mobility of charge +4 as a function (linear fit) of electrophoretic mobility of charge +4.

FIG. 21 graphs 1/K0 as a function of m/z for +1, +2, +3, and +4 charge states.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

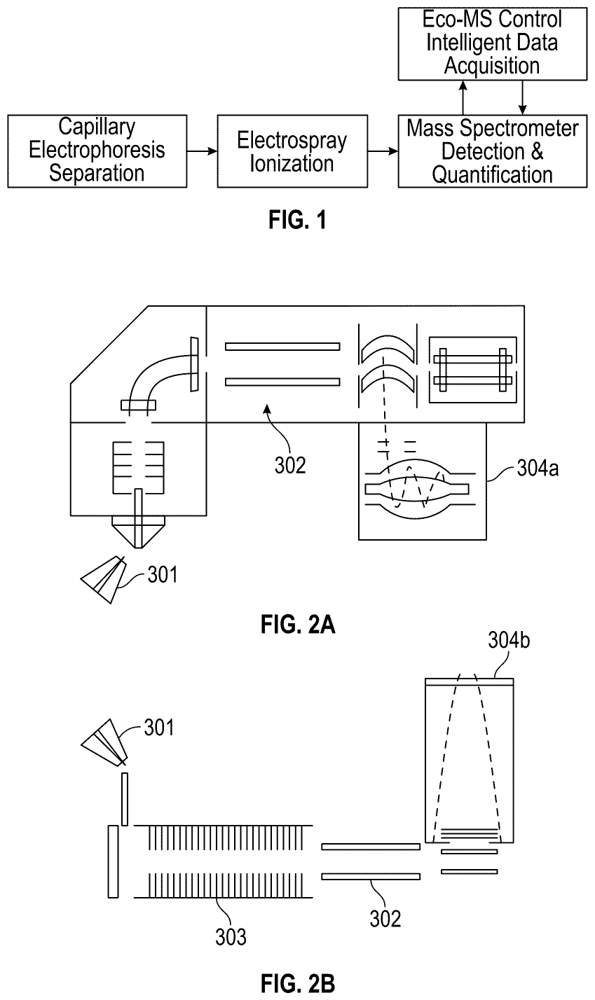

Mass spectrometry (MS) is the leading technology to identify and quantify molecules based on the detection of the mass-to-charge (m/z) ratio of the ions that they generate. MS is the primary driver of proteomics, peptidomics, and metabolomics, as well as the analysis of post-translational modifications. As illustrated in FIG. 1, MS determines the mass, specifically the m/z ratio of ions in exquisite accuracy and precision, down to milli-Daltons and submilli-Daltons at present. For MS analysis, molecules are converted to gas-phase ions, usually using electrospray ionization (ESI), by attaching or removing n number of cations to generate ions of n+ or n− charge states, respectively. These ions are detected and quantified in the mass spectrometer.

As illustrated in FIG. 2, various types of mass spectrometers exist that use different principles of operation, ranging from time-of-flight (TOF) to orbitrap detection. Recent developments have also made MS capable of separating ions in the gas phase based on differences in their collision cross section (CCS) using a technology called ion mobility (IM) spectrometry (IMS), thus enhancing molecular identification and quantification. After recording the m/z value of the detected ions using a survey (aka single-stage or MS1) scan, a portion of the available ions is selected for fragmentation. These ions are isolated based on m/z or ion mobility in specialized compartments called the “isolation cell” using ion optics (e.g., quadrupoles, hexapoles, ion funnel) or ion trapping (e.g., 2- and 3-dimension ion traps and IM trap). To identify molecules, their ions (called molecular ions) are isolated in an isolation cell based on m/z, fragmented in a dissociation cell, and the resulting fragment ions are detected in the analyzer-detector system. Diverse types of fragmentation technologies exist and are used routinely on mass spectrometers.

How a mass spectrometer executes the data-acquisition method fundamentally determines the types and numbers of molecular ions that can be detected and quantified. To enable molecular identifications and quantification, the ions are isolated based on m/z (e.g., in a quadrupole isolation cell) or ion mobility (e.g., in an IM cell) and fragmented into finger-print-like spectra via tandem MS (also known as MS2 or MS/MS) or multi-stage MS (also known as MSn) in the fragmentation cell (e.g., in the CID, HCD, ETD cell).

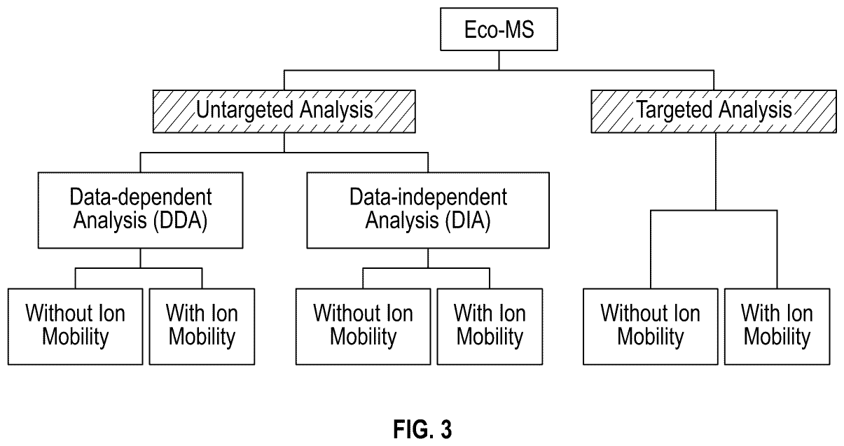

As partially illustrated in FIG. 3, there are three primary ways at present to operate MS/MS:
a) In data-dependent analysis (DDA), ions are selected within a narrow isolation m/z window (e.g., ±1 Da) to enhance molecular specificity and prioritized for fragmentation based on signal abundance. This method inherently is challenged by the number of MS/MS events that a mass spectrometer can execute in a limited time, called the duty cycle (or bandwidth) of DDA and the abundance of the selected ions (called precursor ions).
b) In data-independent analysis (DIA), ions spanning a broad m/z range (called wide-isolation window) are selected for fragmentation altogether (e.g., all ions between 500-525 Da). The mass spectrometer is programmed to scan multiple wide isolation windows to cover large ranges of m/z values. As all the precursor ions within each isolation window are theoretically selected and fragmented in DIA, this MS/MS approach elevates the duty cycle of molecular identifications, thus improving the sensitivity of the detectable proteome.
c) In targeted data analyses, a list of ions of specific m/z values are measured. The m/z values of interest are cycled. Optionally, the time of separation is also considered to further enhance molecular specificity. This approach inherently reduces the coverage of the detectable proteome, is limited to fewer molecular ions, albeit significantly advances quantification sensitivity.

Scientists are in pursuit of better ways to operate mass spectrometers to detect and quantify more molecules at higher sensitivity. A key limitation at present arises from the limited number of analyses that result due to limitations in MS/MS “duty cycle” using DDA, DIA, or targeted analyses. This limitation in MS/MS (or MSn) bandwidth fundamentally challenges molecular identifications, such as that of proteins and peptides, which are key molecules of interest in MS measurements. There is a high and still unmet need to develop new tools, specifically data acquisition methods, to operate mass spectrometers with a capability to detect deeper proteomes and in increased sensitivity.

To enhance detection, biomolecules are usually separated before detection so that their molecular ions are introduced into the mass spectrometer for analysis slowly, ideally one after the other. Because mass spectrometers can identify only so many molecules at a given time due to the aforementioned limitation in MS/MS (MSn) duty cycle, chemical separation dramatically enhances the depth of molecular coverage. Currently, liquid chromatography (LC) is broadly used for separation at various flowrates and configurations, called high-performance LC (HPLC), ultrahigh-performance LC (UPLC), or nano-flow LC (nanoLC). LC retains molecules based on their hydrophobic/hydrophilic characteristics on a stationary phase. Capillary electrophoresis (CE) is an alternative technology, which separates molecular ions under an electric field based on differences in their electrophoretic mobility. The mechanism of separation by LC and CE are fundamentally different.

Several strategies have been developed to improve the sensitivity of proteome detection, both using LC and CE. A priori knowledge of separation times allowed for targeted analysis of peptides, thus enhancing protein detection using LC-MS. Artificial neural network-based methods were developed to predict peptide separation for proteomics. In CE-MS, multi-dimensional separation was developed to benefit MS/MS duty cycle (S. B. Choi, P. Nemes et al., J. Am. Soc. Mass Spectrom. 2017, 29, 913-922, DOI: 10.1007/s13361-017-1838-1, which is hereby incorporated by reference in its entirety herein) and signal abundance based prioritization of peptides reduced redundant analyses (S. B. Choi, P. Nemes et al., Anal. Chem. 2021, 93, 15964-15972, DOI: 10.1021/acs.analchem.1c03327, which is hereby incorporated by reference in its entirety herein), thus deepening the detectable proteome.

A specialized data acquisition method called multiNotch isolation was introduced to synchronously isolate ions to enhance sensitivity and the fidelity of peptide identifications. This multiNotch data acquisition method has been a popular and leading feature on nearly all tribrid Thermo mass spectrometers since its introduction. There is a critical and still unmet need to develop next-generation data acquisition methods to substantially enhance the sensitivity of commercial MS instruments.

To advance MS identification and quantification of molecules, disclosed herein is a next-generation data-acquisition method called “Electrophoresis-correlative mass spectrometry,” shortened as Eco-MS. Illustrative embodiments of the present disclosure are described for proteomics, viz. the analysis of proteins, although embodiments of the present disclosure are also amenable to other types of molecules (e.g., metabolites, transcripts, etc.). As illustrated in FIG. 1, Eco-MS drives the mass spectrometer's operation to improve proteomics analyses. It is noted that mass spectrometers can be programmed to execute “data acquisition methods” that specify how ions are analyzed in the analyzer of a mass spectrometer system (FIGS. 2A-2B). As illustrated in FIG. 3, Eco-MS intelligently executes DDA, DIA, or targeted data acquisition to enhance detection sensitivity and quantification.

CE separates ions based on differences in their electrophoretic mobility in relation to the mass of the ions, forming mass (Daltons) vs. migration time (MT) trends for various charge states (C. Lombard-Banck, P. Nemes et al, Mol. Cell. Prot. 2016, 15, 2756-2768, DOI: 10.1074/mcp.M115.057760, which is hereby incorporated by reference in its entirety).

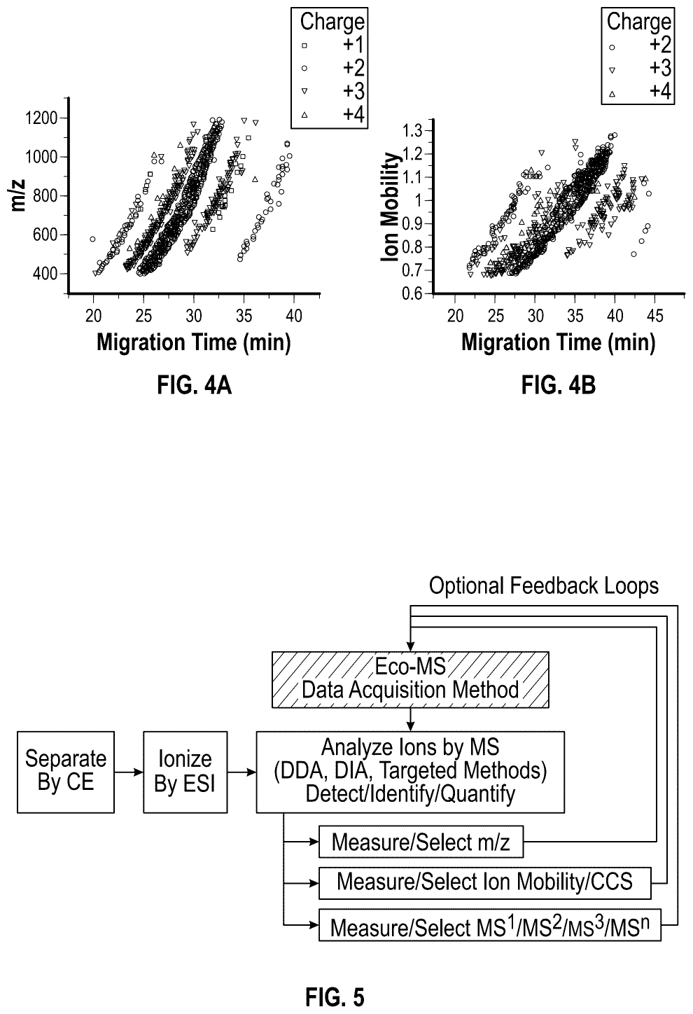

Demonstrated and recognized herein is that CE also orders ions into m/z series over MT (FIG. 4A), thereby ions into trends in mass-to-charge (m/z) ratio vs. separation time for each different n charge state (+n shown in FIG. 4A). Further demonstrated and recognized herein is that CE-separated ions also form trends in relation to their IM measured by IMS, forming trend lines in IM vs. MT for each different charge state (FIG. 4B).

According to one or more aspects of the present disclosure, and as shown in FIG. 3, the Eco-MS data acquisition method leverages these correlations in m/z vs. MT to enable intelligent DDA. The conventional DDA method prioritizes ions for detection based on their signal abundance, which in turn randomizes the m/z values that need to be isolated (e.g., in the quadruple), thus further taxing utilization of the limited MS/MS (MSn) duty cycle to scan over a broad m/z range. In addition, this process is sensitive to abundant contaminant ion signals, which also become selected for analysis in conventional DDA. In contrast, Eco-MS uses an m/z vs. MT correlation to narrow at any time of separation the m/z range where ions need screening based on abundance for MS/MS (or MSn), thus enhancing the overall utilization of the limited sequencing duty cycle of the mass spectrometer. Further, an m/z vs. MT correlation allows Eco-MS to also distinguish, essentially filter out contaminant ions outside m/z vs. MT trend formed by peptides.

According to one or more aspects of the present disclosure, and as shown in FIG. 3, the Eco-MS data acquisition method leverages these correlations in IM vs. MT to enable intelligent DDA using IM mass spectrometry. In this scenario, the m/z vs. MT correlation and IM vs MT correlation allow Eco-MS to narrow ion screening both in the m/z and IM domains of analyses, thus improving the operation of both the ion filter (e.g., quadrupole isolation cell) and the IM cell. This strategy therefore enhances molecular specificity, filters out contaminant ions, and improves the utilization of the sequencing duty cycle.

According to one or more aspects of the present disclosure, and as shown in FIG. 3, the Eco-MS data acquisition method leverages these correlations in m/z vs. MT to enable intelligent DIA. In the conventional DIA data acquisition method, multiple ions within a wide m/z isolation window are isolated (e.g., in the quadrupole isolation cell) and fragmented, and this wide m/z window is scanned over the entire m/z range of analysis. In LC, where molecules are separated based on hydrophobicity rather than size, the m/z values that need detection emerge in a randomized fashion over separation time, thus challenging the duty cycle of wide-window DIA scans seeking to cover the entire m/z range. In contrast, Eco-MS leverages m/z vs. MT correlation to essentially sort m/z values over separation time, which is paralleled by directional scanning of wide-m/z window analyzing the entire m/z range during DIA. Therefore, this natural match between ion m/z sorting by CE and m/z scanning by DIA enhances the overall duty cycle of analyses. As Eco-MS-driven DIA focuses tandem MS acquisition onto peptides within m/z vs. MT trends, the resulting wide-m/z-window MS/MS spectra become less complex, which help better sequence and identify peptides and proteins.

According to one or more aspects of the present disclosure, and as shown in FIG. 3, the Eco-MS data acquisition method leverages these correlations in IM vs. MT to enable intelligent DIA using IM mass spectrometry. In this scenario, the m/z vs. MT and IM vs. MT correlation are used by Eco-MS to respectively structure ion selection during DIA scanning of the wide-m/z-windows and the IM region. This strategy therefore enhances molecular specificity, filters out contaminant ions, and improves the duty cycle utilization of both IM filtration and MS/MS.

According to one or more aspects of the present disclosure, and as shown in FIG. 3, the Eco-MS data acquisition method leverages these correlations in m/z vs. MT to enable intelligent targeted analyses. In traditional targeted analyses, a targeted set of m/z values are cycled for MS/MS. A correlation in m/z vs. MT in Eco-MS allows the mass spectrometer to enhance duty cycle utilization for the quadrupole isolation cell, thus benefiting operation of the hyphenated mass analyzer. This strategy therefore enhances sensitivity and quantification.

According to one or more aspects of the present disclosure, and as shown in FIG. 3, the Eco-MS data acquisition method leverages these correlations in IM vs. MT to enable intelligent targeted analyses using IM mass spectrometry. A correlation in IM vs. MT in Eco-MS allows the mass spectrometer to enhance duty cycle utilization for both the quadrupole isolation cell and IM filtration, thus benefiting operation of the hyphenated mass analyzer. This strategy therefore enhances both molecular specificity and detection sensitivity and quantification.

Eco-MS may be implemented on broad types of mass spectrometers. The mass spectrometers may include, but are not limited to, quadrupole, ion trap, orbitrap, and time-of-flight mass analyzers as well as IM instruments. FIG. 1 presents the general approach to implementing Eco-MS on any mass spectrometer, including orbitrap, TOF, and IM mass spectrometers (FIG. 2). FIG. 3 presents applications that Eco-MS advances but should not be viewed as inclusive of all possible embodiments of the present disclosure.

FIG. 5 is a flow-chart of Eco-MS-enhanced detection/identification/quantification of molecular ions using MS. CE separates molecules, ESI converts them to gas-phase ions, and the mass spectrometer detects the resulting molecular ions. Eco-MS leverages m/z vs. MT and/or IM vs. MT correlations to intelligently operate the mass spectrometer to select ions for detection (MS¹), fragmentation for identification (MS², MSⁿ), and quantification (MS¹, MS², MSⁿ). Optionally Eco-MS establishes a feedback loop, whereby the detected information in the m/z, IM, and/or MSⁿ domain is used to progressively refine the operation of the mass spectrometer. The Eco-MS data acquisition method naturally nests into DDA, DIA, and targeted operational modalities executed by the mass spectrometer to enhance molecular detection, identification, and quantification of molecules.

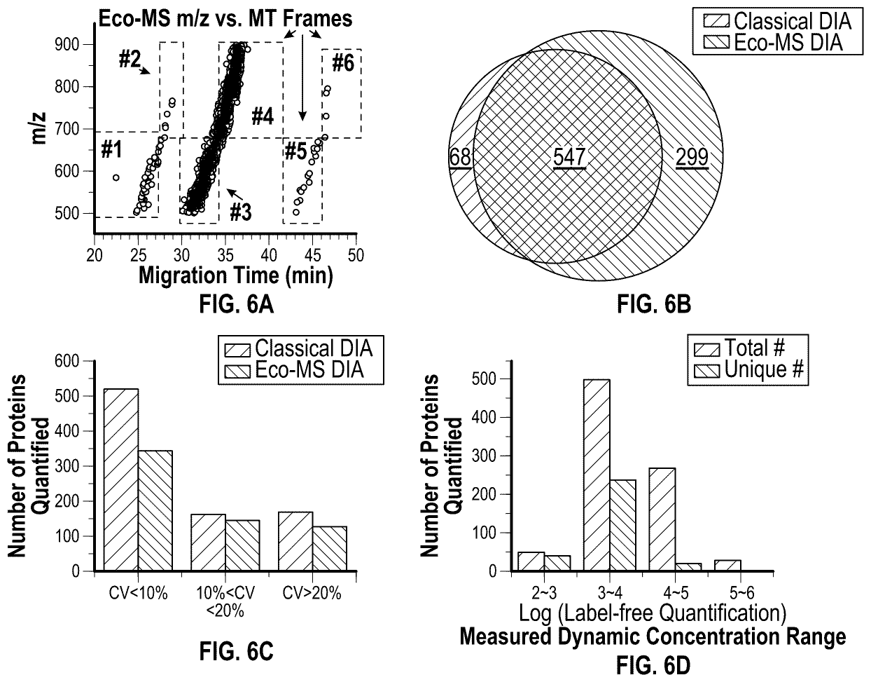

FIGS. 6A-6D demonstrates Eco-MS-enhanced protein identification/quantification from 1 ng of standard HeLa proteome digest (Thermo Fisher) that was analyzed on a Q-Exactive Plus (Thermo Scientific) orbitrap mass spectrometer executing DIA (see FIG. 2A, 304A). As demonstrated in FIG. 4A, ions with charge states ranging from +1 to +4 were detected in the orbitrap analyzer. To enhance the clarity of this presentation, FIG. 6A plots the m/z vs. MT correlation for the +2 peptide ions during CE-ESI analysis. In the classical DIA, ions between the m/z 500-900 range are analyzed using a series of wide-mass isolation windows at every time point of the separation, even when ions are not generated within the measured m/z range.

For example, between 20-30 min of separation, no +2 ions were formed in the m/z 700-900 range, yet this mass range was still scanned using the classical DIA approach. Unnecessary m/z analysis where ions do not exist lowers the efficiency of ion detection. In contrast, using Eco-DIA-MS, this total m/z range becomes dividable into specific and narrower m/z ranges that need scanning over separation time, thus enhancing the efficiency of ion detection.

Eco-MS was used to divide the entire m/z 500-900 range into 6 frames at different separation times (MT values shown in minutes): m/z 500-700 at 20-27 min (frame 1), m/z 700-900 at 27-30 min (frame 2), m/z 500-700 at 30-34 min (frame 3), m/z 700-900 at 34-42 min (frame 4), m/z 500-700 at 42-47 min (frame 5), and m/z 700-900 at 47-50 min (frame 6).

Scanning smaller m/z ranges with wide-isolation DIA scans allows Eco-MS to benefit the DIA method by allowing it to implement smaller DIA m/z-isolation windows without elongating the total cycle time. For example, while the conventional DIA scanned the m/z 500-900 range with 40 rolling windows with each measuring 10 m/z width, the Eco-MS approach allowed for scanning the same net m/z range using 2 Eco-MS frames between 20-27 min of separation, with each using DIA isolation widths of 5 m/z. The success of protein identifications is compared between the classical and Eco-MS-driven DIA in FIG. 6B. While the classical approach reported 615 proteins, Eco-MS identified 846 proteins. Eco-MS allowed to boost protein identifications by ˜38% using 6 different Eco-MS frames. FIG. 6C compares the quantitative performance of the classical and Eco-MS-driven DIA strategies. Eco-MS allowed DIA to quantify more proteins with less error, as reflected in their calculated coefficient of variation (CV) values. Based on the measured label-free quantification abundances, which effectively approximate protein concentration, ˜52% more proteins were quantifiable with <10% CV. FIG. 6D evaluates the sensitivity of the protein detection between the classical and Eco-MS driven DIA approaches. Eco-MS not only identified, but also quantified more proteins. Proteins that were only quantifiable by Eco-MS populated the lower spectrum of the dynamic concentration range, as indicated by more proteins populating lower log (label-free quantification) values.

To a person knowledgeable in the state of art, it is possible to use a higher number (density) of Eco-MS frames to further improve identifications through tailoring of DIA experimental settings to the observed m/z vs. MT trends. Although this example divided m/z vs. MT into only 6 different frames, substantial improvements were realized in protein identification and quantification. In a user-free embodiment of Eco-MS, an automated algorithm continuously rolls even smaller DIA isolation-windows over the observed m/z vs. MT trend lines, essentially closely mapping them, to maximize protein identification and quantification.

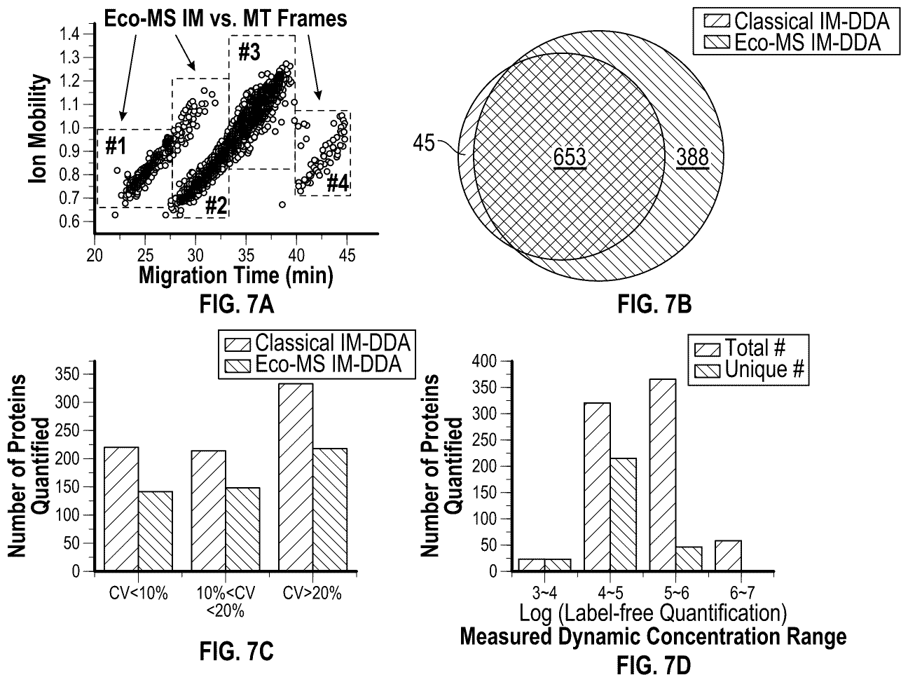

FIG. 7 demonstrates Eco-MS-enhanced protein identification/quantification from 500 μg of standard HeLa proteome digest (Thermo Fisher) that was analyzed on an IM TOF mass spectrometer executing DDA (instrument: trapped IMS or ttimsTOF PRO, Bruker Daltonics, Billerica, MA; see FIG. 2B, IM separation 303 and time-of-flight analyzer 304B). As demonstrated in FIG. 4B, ions with charge states ranging from +2 to +4 were detected in the TOF analyzer. To enhance the clarity of this presentation, FIG. 7A plots the IM vs. MT correlation for the +2 peptide ions during CE-ESI-MS analysis. In classical DDA, ions between the IM 0.60-1.40 range are analyzed by scanning the IM filter at every time point of the separation, even when ions are not generated within the measured IM range. For example, between 20-27 min of separation, no +2 ions were formed with IM 1.0-1.4 region, yet this IM range was still measured, thus wasting the sequencing duty cycle of the instrument. Unnecessary analysis over IM ranges where no ions are present lower the efficiency of ion detection in IMS-MS. In contrast, using Eco-MS, this total IM range becomes divisible into specific and narrower IM ranges that need scanning over specific time points of separation, thus enhancing the efficiency of ion detection.

As an example, Eco-MS was used to divide the entire IM 0.6-1.4 range into 4 frames at different separation times (MT values shown in minutes): IM 0.65-1.00 at 20-27 min (frame 1); IM 0.60-1.20 at 27-32 min (frame 2); IM 0.80-1.40 at 32-40 min (frame 3); and IM 0.70-1.05 at 40-45 min (frame 4). Scanning smaller IM ranges scans allows Eco-MS to better utilize the duty cycle and the ion trapping capability of the IM cell, thus improving DDA. For example, while the conventional IM-DDA scanned the IM 0.60-1.40 range every MT point, the Eco-MS approach allowed for scanning only specific IM ranges (see windows in FIG. 7A). The success of protein identifications is compared between the classical and Eco-MS-driven IM-DDA in FIG. 7B. While the classical approach reported 698 proteins, Eco-MS identified 1,041 proteins. Eco-MS allowed to boost protein identifications by ˜49% improvement than conventional IM-DDA (FIG. 7B). FIG. 7C compares the quantitative performance of the classical and Eco-MS-driven IM-DDA strategies. Generally, Eco-MS allowed DDA to quantify more proteins. For example, Eco-MS quantified 55% more proteins with CV<10%. FIG. 7D evaluates the sensitivity of protein detection between the classical and Eco-MS driven IM DDA approaches. Eco-MS not only identified, but also quantified more proteins across the entire concentration range. Proteins that were only quantifiable by Eco-MS populated the lower domain of the dynamic concentration range, as indicated by more populating lower log (label-free quantification) values.

To a person knowledgeable in the state of art, it is possible to use a higher number of Eco-MS IM vs. MT frames to further improve identifications through tailoring of DDA experimental settings to the observed IM vs MT trends. Although this example divided the whole IM vs. MT range into only 4 parts, Eco-MS IM-DDA delivered substantial improvements. In a user-free embodiment of Eco-MS, an automated algorithm continuously rolls even smaller IM ranges over the observed IM vs. MT trend lines, essentially closely mapping them, to maximize protein identification and quantification.

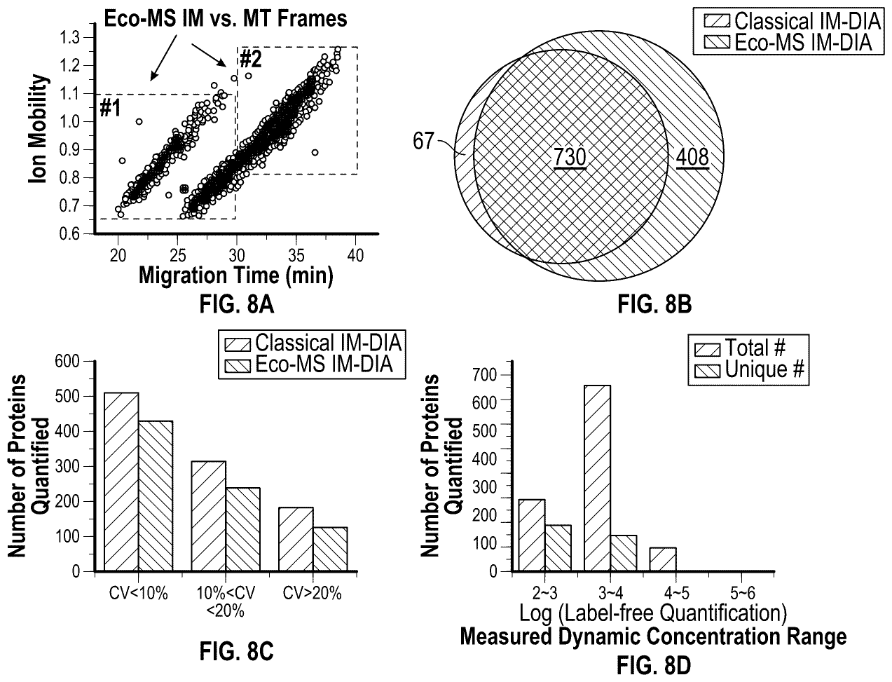

Eco-MS is also amenable to the integration of DIA and IM-MS for deeper proteome detection and quantification. FIGS. 8A-8D demonstrates Eco-MS-enhanced protein identification/quantification from 500 μg of standard HeLa proteome digest (Thermo Fisher) that was analyzed on an IM TOF mass spectrometer executing IM-DIA (instrument: timsTOF PRO, Bruker Daltonics, Billerica, MA; recall FIG. 2B, see IM separation 303 and time-of-flight analyzer 304B). As demonstrated in FIG. 4B, ions with charge states ranging from +2 to +4 were detected in the TOF analyzer.

To enhance the clarity of this presentation, FIG. 8A plots the IM vs. MT correlation for the +2 peptide ions during CE-ESI-MS analysis. In classical IM DIA, ions between the IM 0.60-1.40 range are analyzed by scanning the IM filter at every time point of the separation with a 20 m/z wide-mass isolation window, even when ions are not generated within the measured IM range, thus wasting sequencing efficiency. As earlier (see FIG. 7A), between 20-25 min of separation, no +2 ions were formed with IM 0.90-1.40 region, yet this IM range was still measured in the classical approach. Unnecessary analysis over IM ranges where no ions are present lower the efficiency of ion detection in IMS-MS. In contrast, using Eco-MS, this total IM range becomes divisible into specific and narrower IM ranges that need scanning over specific time points of separation, thus enhancing the efficiency of ion detection. As an example, Eco-MS was used to divide the entire IM 0.6-1.4 range into 2 frames at different separation times (MT values shown in minutes), with each scanning a m/z 15 wide-mass isolation window for DIA: IM 0.63-1.10 at 0-30 min (frame 1) and IM 0.80-1.25 at 30-40 min (frame 2). The success of protein identifications is compared between classical and Eco-MS-driven IM-DIA in FIG. 8B. While the classical approach reported 797 proteins, Eco-MS identified 1,138 proteins, essentially boosting protein identifications by ˜43% improvement than the conventional IM-DIA approach. FIG. 8C compares the quantitative performance of the classical and Eco-MS-driven IM-DIA strategies. Generally, Eco-MS allowed IM-DIA to quantify more proteins while maintaining excellent quantitative reproducibility. FIG. 8D evaluates the sensitivity of protein detection between the classical and Eco-MS driven IM-DIA approaches. Eco-MS not only identified, but also quantified more proteins across the entire concentration range. Proteins that were only quantifiable by Eco-MS populated the lower-middle domain of the dynamic concentration range, as indicated by more proteins populating lower log (label-free quantification) values.

A higher number of Eco-MS IM vs. m/z vs. MT frames can be used to further improve identifications through tailoring of IM-DIA experimental settings to the observed IM vs. m/z vs. MT trends. Although this example only divided the whole IM range into two gross frames, Eco-MS IM-DIA was still able to deliver substantial improvements. In a user-free embodiment of Eco-MS, an automated algorithm continuously rolls even smaller IM vs. m/z vs. MT frames and uses smaller DIA mass-isolation windows over the observed IM vs. m/z vs. MT trend lines, essentially closely mapping them, to maximize protein identification and quantification.

From the foregoing, it can be seen that the present disclosure accomplishes at least all of the stated objectives.

### EXAMPLES

Embodiments of the present disclosure show the idea developed and tested on two instruments, an orbitrap instrument executing data-independent acquisition (DIA) and a timsTOF PRO executing data-dependent acquisition (DDA). Eco-DIA-MS using a Q-Exactive Plus (Thermo) identified 846 proteins from 1 ng of HeLa protein digest via library-free search, achieving ˜38% improvement to the conventional DIA method without m/z prediction. Preliminary experiments demonstrated these improvements to become increasingly valuable for single-cell studies. Through label-free quantification to estimate protein concentration, we found 78% of the exclusively Eco-DIA-MS identified proteins occupied the lower domain of the concentration dynamic range. Furthermore, the adaptability of the approach to an ion mobility separation was tested. Embodiments of the present disclosure include developed Eco-ddaPASEF-MS on a timsTOF PRO (Bruker) instrument, identifying 1,041 protein groups from 500 μg of HeLa protein digest, essentially doubling protein identifications compared to the conventional ddaPASEF method. Low coefficients of variation of the calculated label-free quantitative abundances (˜10-20%) revealed good quantitative reproducibility. These performance metrics suggested a technical capability to study cellular dynamics. Testing Eco-MS on embryonic subcellular contents and mammalian visual system-related tissues, such as the retina and optical nerves, help bring this technology innovation to broad users and scientific backgrounds. The application of Eco-MS can help profile the proteome from limited biological samples and investigate how the changes in proteomic profiles control states of health and disease.

In Eco-DIA-MS, the total scan range (m/z 500-900) was divided into two DIA windows (m/z 500-700 and 700-900). Windows 1 and 2 were applied to the specific separation durations to cover most of the +2-charged precursor ions. The isolation window widths of eco-DIA-MS and conventional DIA-MS were set as m/z 5 and 10, respectively. The conventional DDA parallel accumulation-serial fragmentation (PASEF)-MS was set to scan the complete mobility range (1/k₀ 0.6-1.6), while the eco-ddaPASEF-MS scanned part of the ion mobility range to cover most of the +2 charged precursor ions. The eco-DIA-MS and eco-ddaPASEF-MS were performed with 1 ng and 500 μg of HeLa protein digest, respectively. Raw files were analyzed using Spectronaut (DirectDIA) or MSFragger.

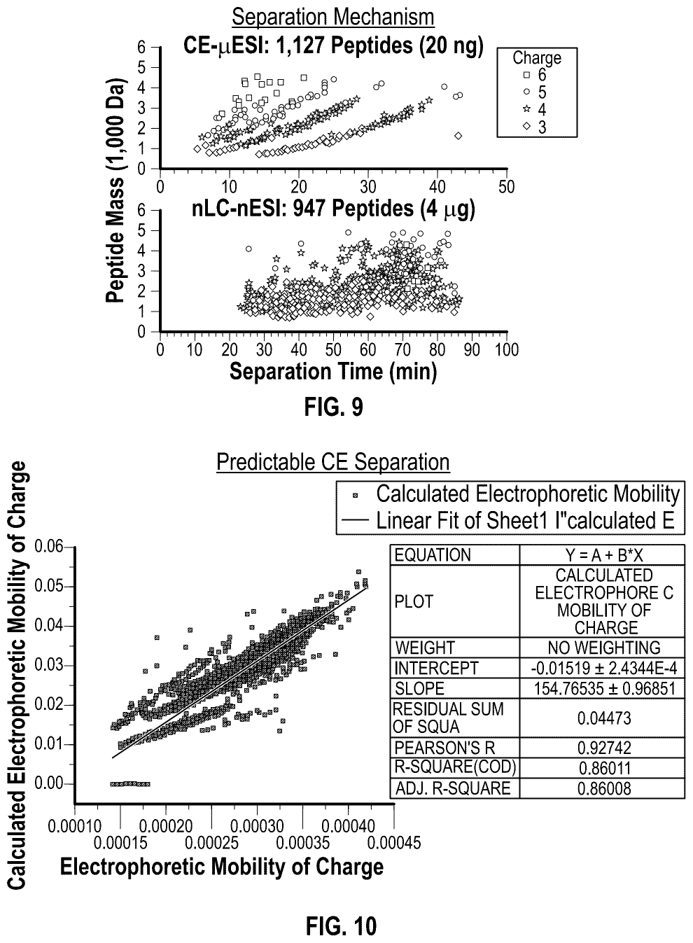

FIGS. 9-10 show that CE separation is different than LC.

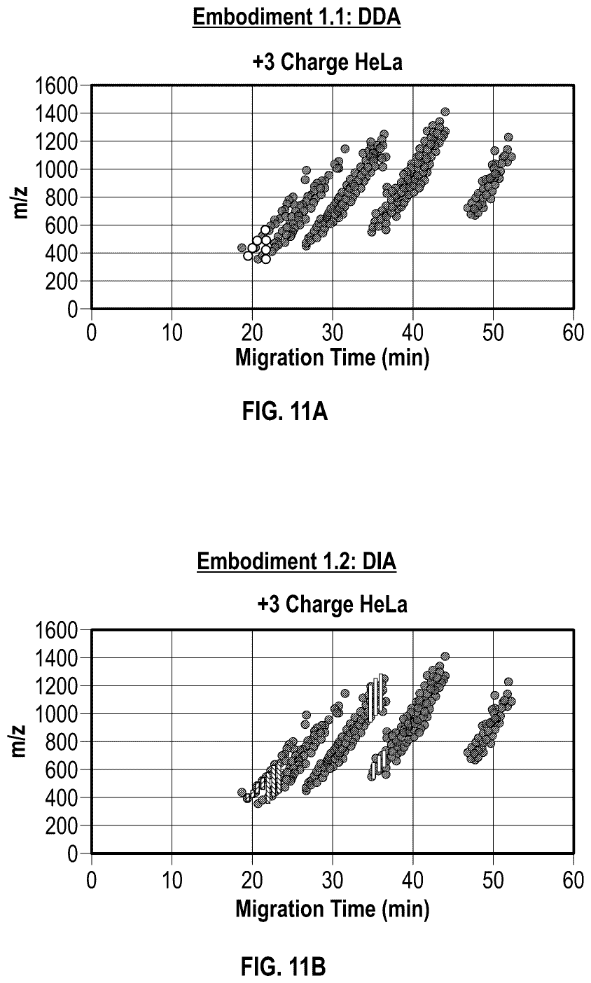

FIGS. 11A-11B show m/z vs. MT correlation in CE to drive “smart” data acquisition for time-of-flight (TOF) instruments. This results in a deeper coverage of the proteome and enables ultrasensitive measurements. FIG. 11A embodies data dependent analysis (DDA). The targeted m/z values with known MT are fragmented. This is analogous to a scheduled precursor, except more logical. FIG. 11B embodies data independent analysis (DIA). Smaller m/z windows are fragmented (higher duty cycle).

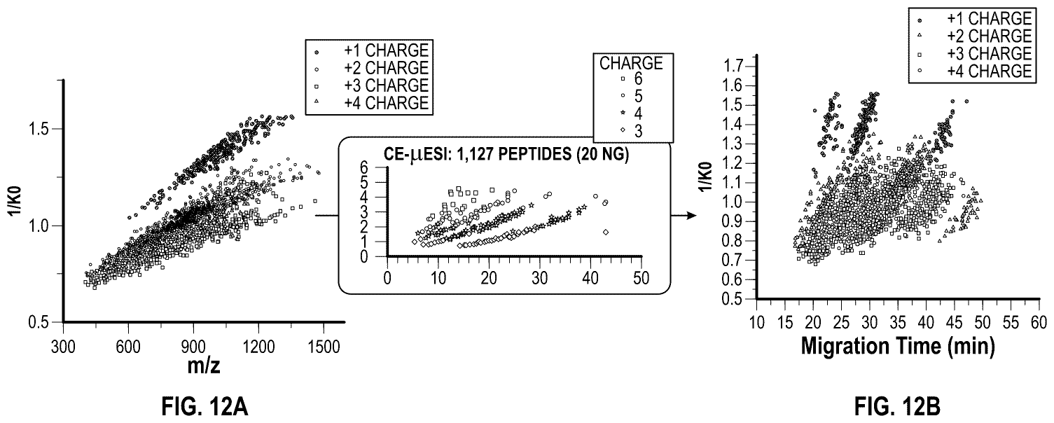

FIGS. 12A-12B show how CE migration correlates with ion mobility (timsTOF PRO).

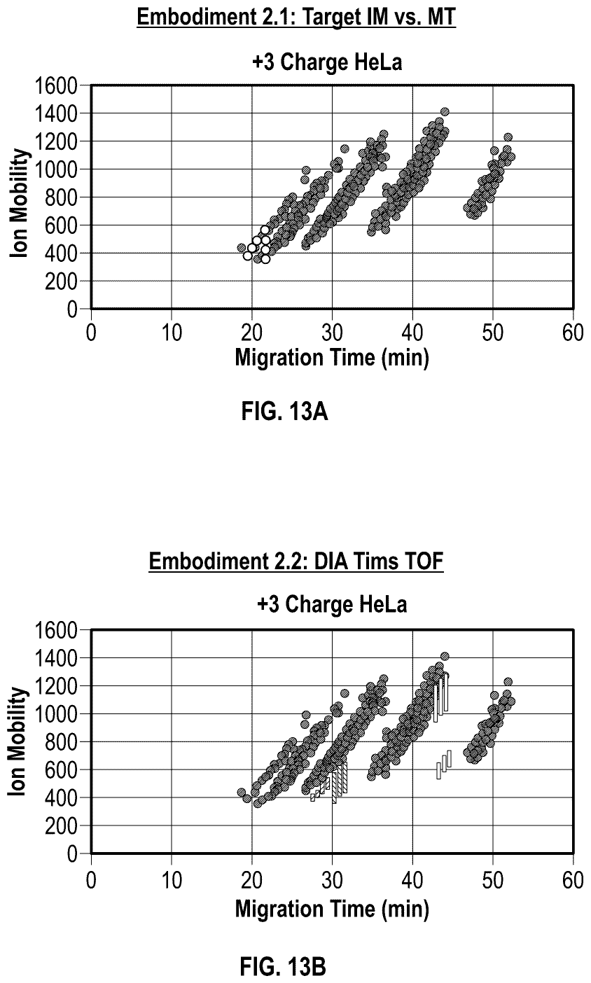

FIGS. 13A-13B shows ion mobility vs. MT correlation in CE to drive “smart” data acquisition for trapped ion mobility spectrometry time-of-flight (timsTOF) instruments. This results in a deeper coverage of the proteome and enables ultrasensitive measurements. FIG. 13A embodies target IM vs. MT. FIG. 13B embodies data independent analysis (DIA) for a trapped ion mobility spectrometry time-of-flight (timsTOF) instrument.

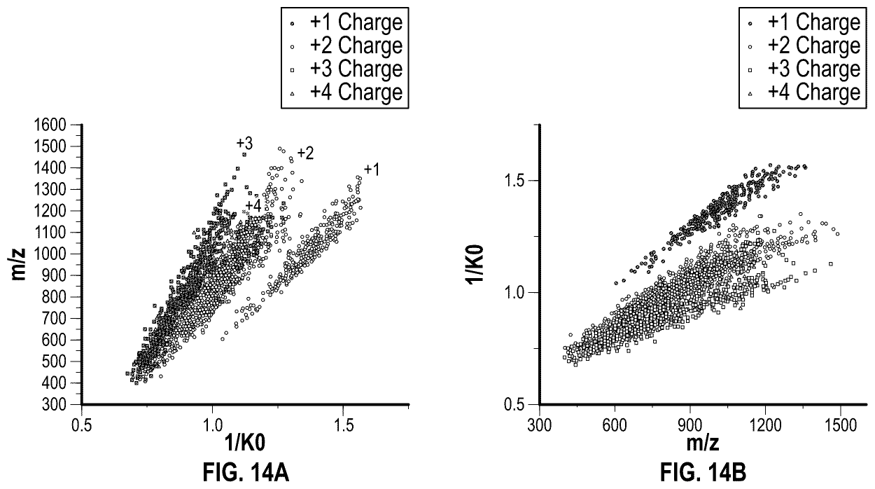

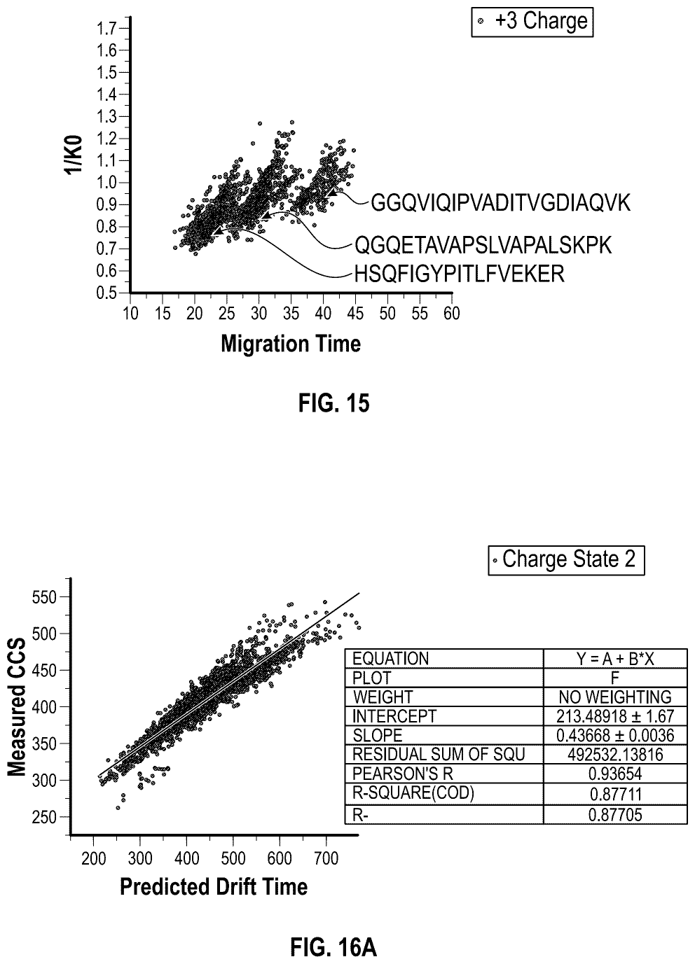

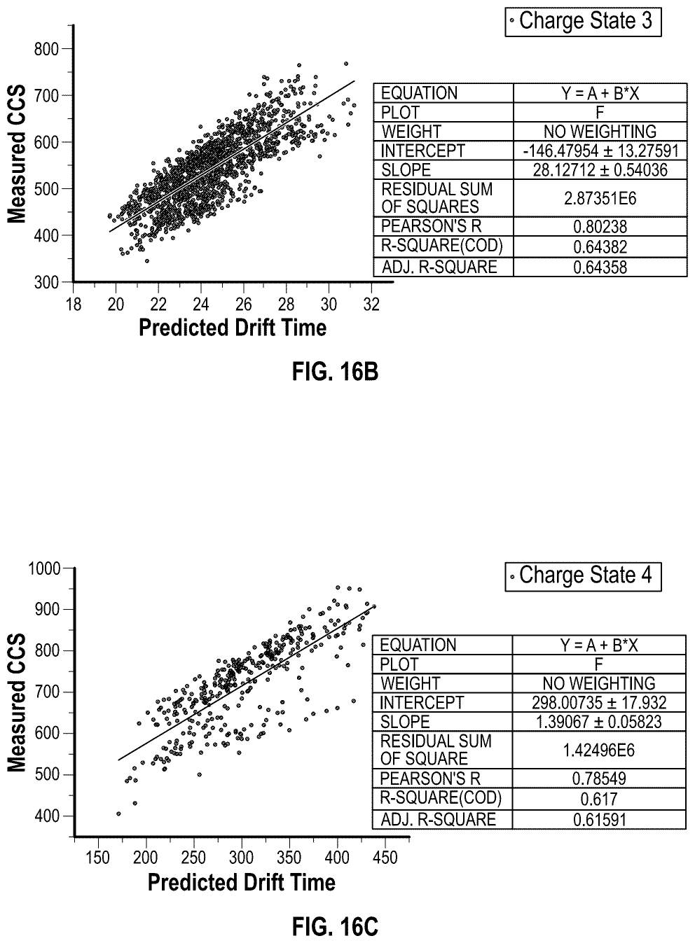

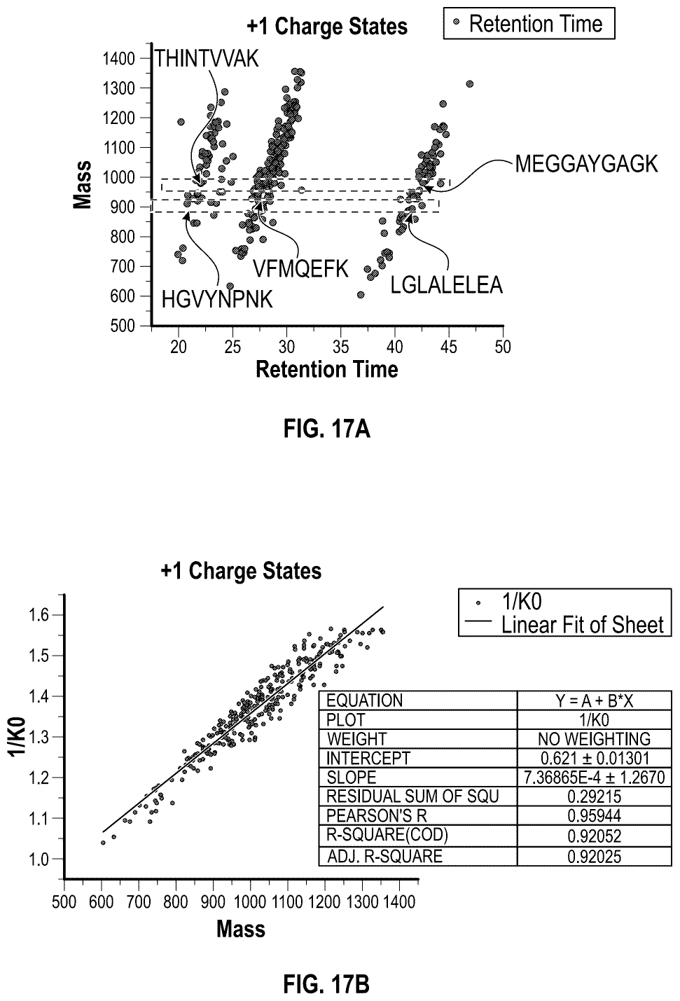

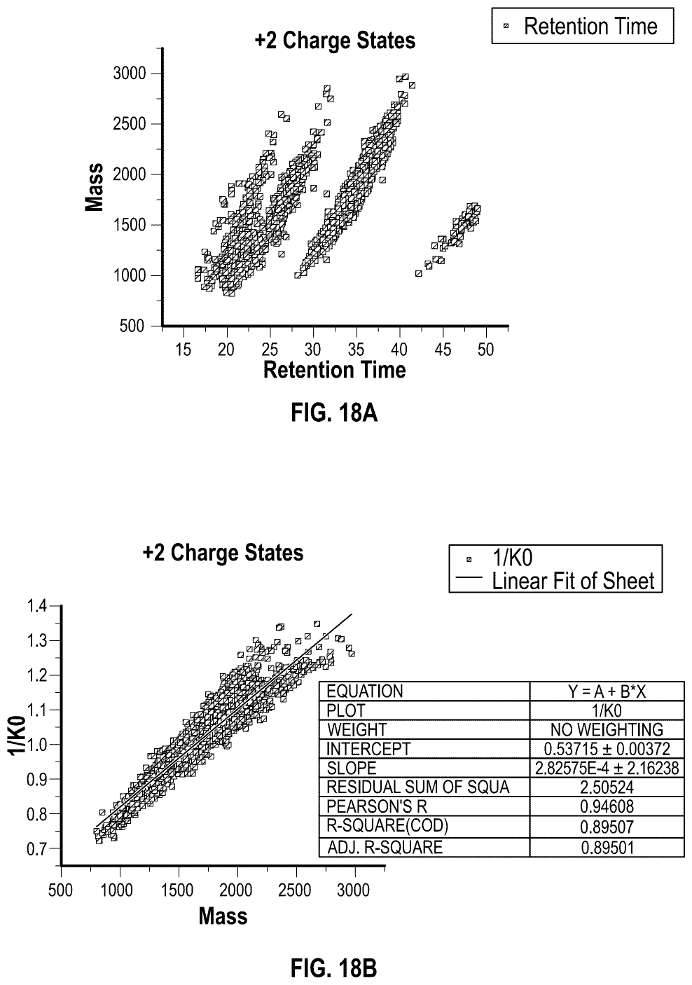

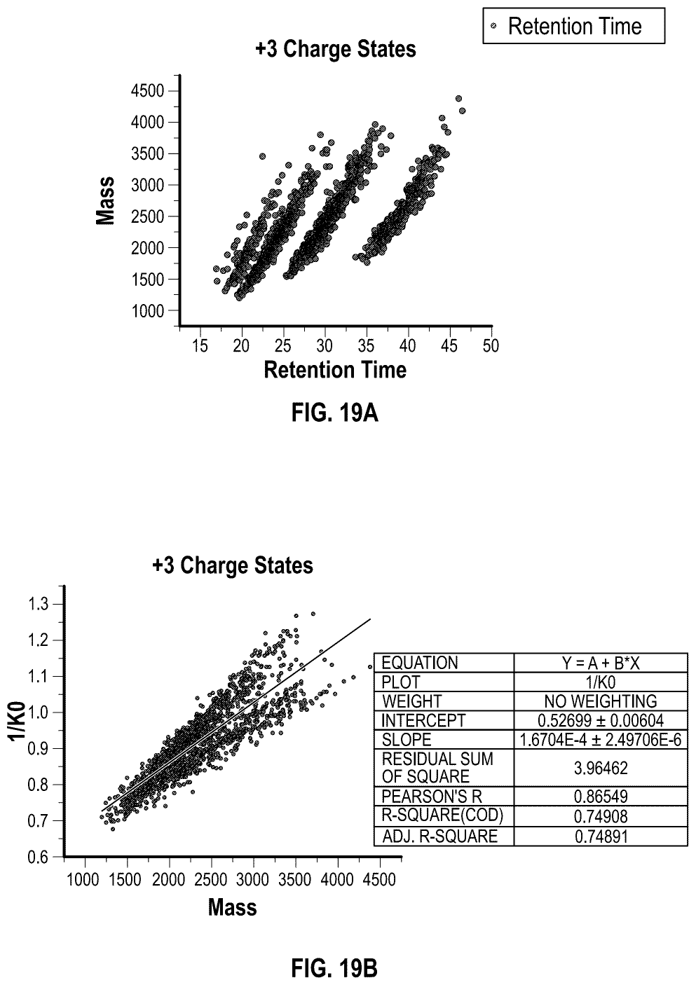

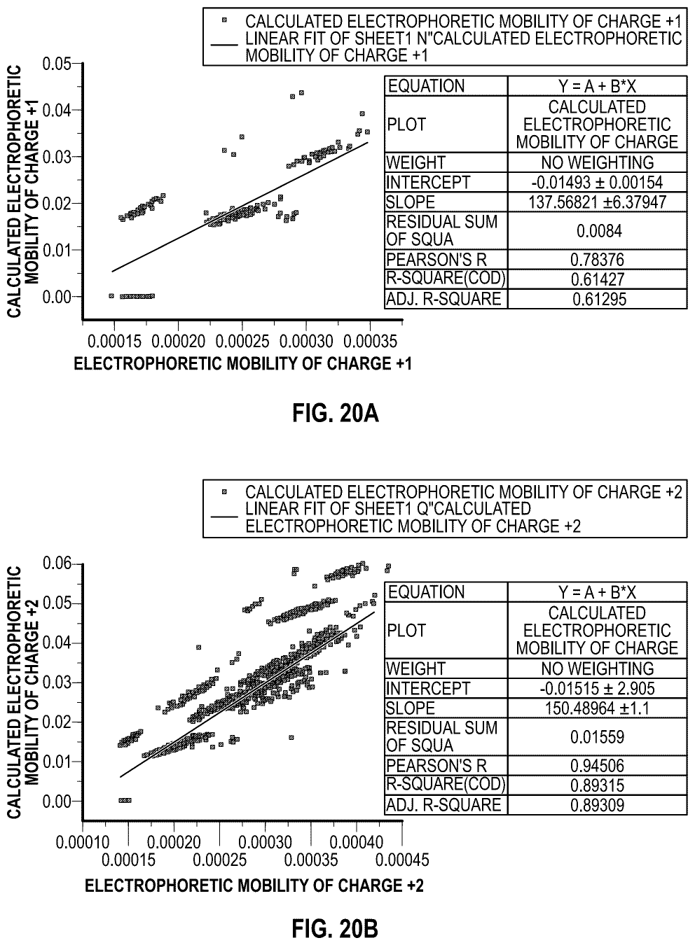

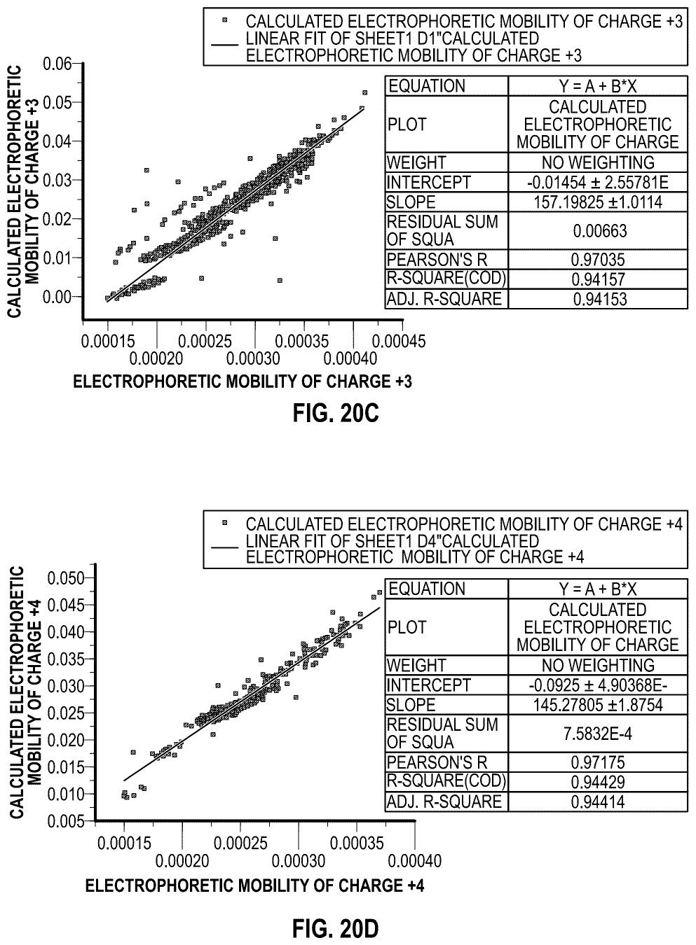

FIGS. 14A-20D show supporting data from a lab with four distinct types of ultrasensitive HRMS platforms: (i) a first generation 25 amol MS (TOF) platform that utilizes CE and CE-μESI; (ii) a second generation 210 zmol HRMS (OT) platform that utilizes CE and CE-nESI; (iii) a third generation HRMS (Q-QT-IT) platform that utilizes CE and CE-nESI; and (iv) a fourth generation trapped ion mobility spectrometry time-of-flight (timsTOF) mass spectrometer that utilizes CE and CE-nESI. CE separates peptides according to some order that matches a trend with their ion mobility. Thus, protocol/methods can be developed to dynamically adjust the K0 parameters to maximize ion accumulation and read-out conditions to enhance sensitivity and quantitative performance. FIGS. 14A-20D show what these trends are, and further, how CE separation time correlates with the ion mobility values (i) generally (for all data irrespective of charge state); and (ii) only for select charge state(s). There is also data for LC-timsTOF.

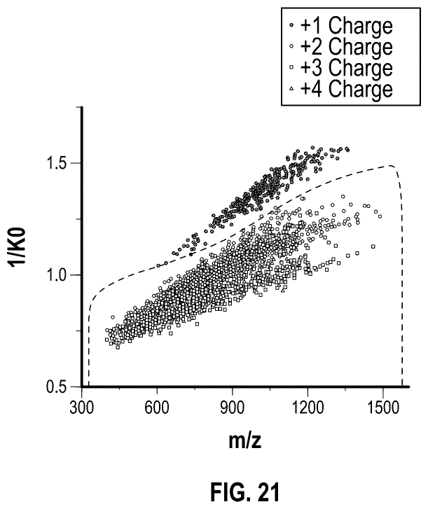

Regarding FIG. 21, there are also peptide groups with different charge states, which would have similar MT with different 1/K0 values. Replicate PSMs were removed from the graph. The peptides are vastly different across the same mobility values with migration time differences. Setting K0 values for ms/ms according to the graph shown in FIG. 21 affects ion accumulation time and quantitative performance.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 301 | ESI-generated ions |
| 302 | quadrupole mass analyzer |
| 303 | IM separation |
| 304A | orbitrap analyzer |
| 304B | time-of-flight (TOF) analyzer |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A method of controlling mass-spectrometry (MS) measurements of a molecule, the method comprising:
using mass to charge ratio (m/z)-dependent separation in capillary electrophoresis (CE) to detect or quantify an aspect of said molecule;
separating ions in the gas phase based on differences in their collision cross section (CCS) using ion mobility (IM) separation (303).

2. The method of claim 1, further comprising producing an ESI-generated ion (301) from said molecule using an electrospray in which a high voltage is applied to a liquid to create an aerosol.

3. The method of claim 1, further comprising:
using a quadrupole of a quadrupole mass analyzer (302) to select ions based on the mass to charge ratio (m/z); and
acquiring data using electrophoresis-correlative mass spectrometry (Eco-MS).

4. The method of claim 1, wherein the use of m/z-dependent separation comprises:
comprises m/z-predictive ion separation to boost an economy of MS proteomics on an orbitrap instrument (304A); or
comprises m/z-predictive ion separation to boost an economy of MS proteomics on a time-of-flight (TOF) instrument (304B) or a trapped ion mobility time-of-flight (timsTOF) instrument;

5. The method of claim 1, wherein the mass to charge ratio (m/z)-dependent separation is known through an electrophoretic mobility of the molecule and said correlation thereto.

6. The method of claim 1, further comprising identifying proteins through a library free search.

7. The method of claim 1, wherein the molecule is selected from the group consisting of: proteins, metabolites, peptides, transcripts, and genes.

8. The method of claim 1, further comprising focusing a mass spectrometer's bandwidth for a specific detection.

9. The method of claim 1, further comprising using migration-predictive correlation to operate a mass spectrometer using an identification strategy.

10. The method of claim 9, wherein:
(i) the identification strategy comprises untargeted analysis;
(ii) the untargeted analysis is data-dependent analysis (DDA)
(iii) an algorithm predicts separation times in the DDA so that the mass spectrometer can be programmed to a non-zero and a non-whole fraction of ions;
(iv) a prediction of the separation times is dynamic.

11. The method of claim 9, wherein:
(i) the identification strategy comprises untargeted analysis;
(ii) the untargeted analysis is data-independent analysis (DIA); and
(iii) predicting a range of m/z is targeted for analysis using DIA.

12. The method of claim 9, further comprising:
(i) using targeted analysis to analyze the aspect of the molecule; and
(ii) the targeted analysis is accomplished with ion mobility (IM).

13. The method of claim 1, further comprising aiding a modality of MS operation selected from the group consisting of: bottom-up proteomics, middle-down proteomics, and top-down proteomics.

14. The method of claim 1, further comprising narrowing a time of separation of the m/z range where ions need screening based on abundance for MS/MS or MS″.

15. The method of claim 1, further comprising filtering out contaminant ions outside m/z vs. MT correlation.

16. The method of claim 1, further comprising:
directional scanning of a m/z window analyzing the entire m/z range during data-independent analysis (DIA); and
using Eco-MS to structure ion selection during DIA scanning of the m/z-window and an ion mobility (IM) region.

17. The method of claim 1, further comprising enhancing duty cycle utilization (i) for a quadrupole isolation cell, thereby benefiting operation of a hyphenated mass analyzer or (ii) for ion mobility (IM) filtration.

18. The method of claim 1, further comprising enhancing molecular specificity, a detection sensitivity, and a quantification ability of an existing mass spectrometer.

19. A mass spectrometer comprising:
a capillary electrophoresis (CE)-based ionizer capable of utilizing electrospray ionization (ESI) to produce ions;
an ion mobility cell that analyzes said ions with electrophoresis-correlative mass-spectrometry (Eco-MS); and
an electrophoresis-correlative mass spectrometry (Eco-MS)-based data acquisition system (DAQ);
wherein the Eco-MS-based DAQ comprises a feedback loop to analyze said ions and the feedback loop further comprises measuring or selecting a parameter from the group consisting of:
a mass to charge ratio (m/z);
an ion mobility (IM);
a collision cross section (CCS);
an ion for detection (MS¹);
a fragmentation for identification (MS², MSⁿ); and
a quantification (MS¹, MS², MSⁿ);
wherein the Eco-MS data acquisition method naturally nests into data-dependent analysis (DDA), data-independent analysis (DIA), and targeted operational modalities executed by the mass spectrometer to enhance molecular detection, identification, and quantification of molecules.

20. An ultrasensitive high resolution mass spectrometry (HRMS) platform comprising:
a mass spectrometer that utilizes using mass to charge ratio (m/z)-dependent separation in capillary electrophoresis (CE) to detect or quantify an aspect of a molecule; and
an electrophoresis-correlative mass spectrometry (Eco-MS)-based data acquisition system (DAQ);
wherein:
the ultrasensitive HRMS platform comprises an attomole (amol)-scale MS (TOF) platform that utilizes CE and CE-μESI;
the ultrasensitive HRMS platform comprises a zettamole (Zmol)-scale HRMS (OT) platform that utilizes CE and CE-nESI;
the ultrasensitive HRMS platform comprises an HRMS (Q-QT-IT) platform that utilizes CE and CE-nESI; or
the ultrasensitive HRMS platform comprises a trapped ion mobility spectrometry time-of-flight (timsTOF) mass spectrometer.
